(19) United States
(12) Patent Application Publication
(10) Pub. No.: US 2024/0306959 A1
(43) Pub. Date: Sep. 19, 2024

(54) SYSTEMS, DEVICES, AND METHODS FOR PERFORMING TRANS-ABDOMINAL FETAL OXIMETRY AND/OR TRANS-ABDOMINAL FETAL PULSE OXIMETRY USING DC OXIMETRY MEASUREMENTS

(71) Applicant: Raydiant Oximetry, Inc., San Ramon, CA (US)

(72) Inventors: Neil Padharia Ray, Sacramento, CA (US); Rodney P. Chin, Rodeo, CA (US); Mark Andrew Rosen, Piedmont, CA (US); Russell DeLonzor, San Ramon, CA (US)

(21) Appl. No.: 18/576,671

(22) PCT Filed: Sep. 2, 2022
(86) PCT No.: PCT/US22/42554

§ 371 Date: Jan. 4, 2024

Related U.S. Application Data
(60) Provisional application No. 63/218,883, filed on Jul. 6, 2021.

(51) Int. Cl. A61B 5/1464; A61B 5/00; A61B 5/1455

(52) U.S. Cl. CPC A61B 5/1464; A61B 5/14552; A61B 5/7275; A61B 2562/0238

(57) ABSTRACT

Analysis of a transabdominally-obtained composite non-pulsatile, or DC signal, for a pregnant mammal and her fetus may provide an indication of a hemoglobin oxygen saturation level for the fetus' non-pulsatile, or venous, blood. This analysis of the composite DC signal may allow for the determination of a relative and/or absolute indication of fetal hemoglobin saturation level for non-pulsatile, or venous, blood without requiring analysis of a fetal pulsatile, or AC, signal.

### RELATED APPLICATION

This application is an INTERNATIONAL patent application that claims priority to U.S. Provisional Patent Application No. 63/218,883 filed 6 Jul. 2021 and entitled “SYSTEMS, DEVICES, AND METHODS for performing trans-abdominal fetal oximetry and/or trans-abdominal fetal pulse oximetry using DC OXIMETRY MEASUREMENTS,” which is incorporated herein in its entirety.

### FIELD OF INVENTION

The present invention is in the field of medical devices and, more particularly, in the field of trans-abdominal fetal oximetry and trans-abdominal fetal pulse oximetry.

### BACKGROUND

Oximetry is a method for determining the oxygen saturation of hemoglobin in a mammal's blood. Typically, 90% (or higher) of an adult human's hemoglobin is saturated with (i.e., bound to) oxygen while only 30-60% of a fetus's blood is saturated with oxygen. Pulse oximetry is a type of oximetry that uses changes in blood volume through a heartbeat cycle to internally calibrate hemoglobin oxygen saturation measurements of the arterial blood.

Current methods of monitoring fetal health, such as monitoring fetal heart rate, are inefficient at determining levels of fetal distress and, at times, provide false positive results indicating fetal distress that may result in the unnecessary performance of a Cesarean delivery.

### SUMMARY

Systems, processes, and/or methods for determining an indication of a fetal venous hemoglobin oxygen saturation level may be configured and/or programmed so that a processor may receive a digital signal corresponding to an optical signal emanating from a pregnant mammal's abdomen. On some occasions, the optical signal is obtained transabdominally via projecting the optical signal into the pregnant mammal's abdomen. The digital signal may be received from, for example, a photodetector proximate to and/or in physical contact with the pregnant mammal's abdomen and/or an analog to digital converter communicatively coupled to the photodetector and processor. The analog to digital converter may be configured to convert a detected photon and/or optical signal (which may be represented as, for example, an analog voltage when detected by the photodetector) into a digital signal that may be analyzed or otherwise processed by a processor, controller, and/or computer.

It may then be determined whether a fetal pulsatile signal is present in the digital signal and, if so, an indication of a fetal venous hemoglobin oxygen saturation level may be determined and communicated to a user via, for example, a display device communicatively coupled to the processor. In some embodiments, determining whether a fetal pulsatile signal is present in the digital signal includes determining whether an intensity of the fetal pulsatile signal is above a threshold intensity value. Determining whether the fetal pulsatile signal is present in the digital signal may provide confirmation that the digital signal at least partially corresponds to light that was incident on the fetus and/or contains a fetal venous or DC signal component.

At times, determining the indication of the fetal venous hemoglobin oxygen saturation level may include analyzing the digital signal and comparing a result of the analysis with a threshold value for the fetal venous hemoglobin oxygen saturation level, wherein the indication of the fetal venous hemoglobin oxygen saturation level includes a result of the comparison to the user. In some embodiments, determining of the fetal venous oximetry level includes performing, for example, a magnitude, average, and/or trend analysis that determines changes in the fetal venous oximetry level over time.

On some occasions, the digital signal may include a composite venous signal that comprises venous signals for both the pregnant mammal and a fetus within the pregnant mammal's abdomen. In these instances, the digital signal may be deconvolved and/or filtered to separate out the maternal and fetal venous signals and or isolate the fetal venous signal from the maternal venous signal. In some embodiments, the maternal digital signal may, on some occasions, correspond to an optical signal incident only on maternal tissue (i.e., the optical signal does not penetrate the abdominal tissue to be incident on the fetus such as a short separation signal. In these embodiments, the maternal venous signal (as understood from, for example, the optical signal incident only on maternal tissue) may be subtracted from the composite venous signal and the resultant venous signal may be the fetal venous signal. Once the fetal venous signal is isolated from the composite venous signal, the fetal venous signal may be further analyzed and/or processed to determine a fetal venous hemoglobin oxygen saturation level.

In some embodiments, the digital signal may be processed and/or filtered (e.g., bandpass, Kalman, etc.) to isolate a fetal venous signal from the digital signal and/or a composite venous signal included in the digital signal prior to determining the indication of the fetal venous hemoglobin oxygen saturation level.

In some embodiments, an indication of changes to a maternal venous signal included in the digital signal over a period of time (e.g., 10 s, 30 s, 1 minute, 10 minutes, etc.) may be received and the digital signal may be processed to remove (e.g., filter and/or subtract) a maternal venous component of the digital signal and the resultant signal may be a fetal non-pulsatile, venous, and/or DC signal. The processed digital signal may then be analyzed to determine changes in the fetal venous signal and/or the processed digital signal over the period of time to determine a relative fetal venous oxygen saturation level, an average fetal venous oxygen saturation level, and/or trend of changes to the fetal venous oxygen saturation level over time. The changes to the fetal venous oxygen saturation level and/or trend analysis may be provided to the user as, for example, a numerical value and/or a graph.

Additionally, or alternatively, systems, processes, and/or methods for determining an indication of a fetal venous hemoglobin oxygen saturation level may be configured and/or programmed so that a processor may receive a composite DC signal corresponding to an optical signal emanating from a pregnant mammal's abdomen. On some occasions, the optical signal is obtained transabdominally via projecting the optical signal into the maternal abdomen. The DC signal may be received from, for example, a photodetector proximate to and/or in physical contact with the pregnant mammal's abdomen and/or an analog to digital converter communicatively coupled to the photodetector and processor. The composite DC signal may then be analyzed. This analysis may include, but is not limited to, performing a trend analysis that determines relative changes in the composite DC signal over time, and/or comparing a result of the analysis with a threshold value for the composite DC signal. An indication of fetal hemoglobin oxygen saturation level may then be determined based on a result of the analysis of the composite DC signal. The indication may then be provided to the user via, for example, a display device in communication with the processor making the determination.

In some embodiments, an indication of changes to a maternal DC signal over a period of time may be received and the composite DC signal may be processed to remove changes to a maternal component of the composite DC signal. The resulting signal may be a processed composite DC signal. The processed composite DC signal may then be analyzed to determine changes in the processed composite DC signal over the period of time so that, for example, a magnitude, average, and/or trend of changes to the processed composite DC signal over time may be determined and provided to the user. In some embodiments, an indication (e.g., present/not present, fetal signal of sufficient strength, etc.) of a fetal AC signal may be received prior to receipt of the composite DC signal and it may be determined whether an intensity of the fetal AC signal is above a threshold intensity value. Continuing to analyze the composite DC signal to determine the fetal venous hemoglobin oxygen saturation level may be responsive to a determination that the intensity of the fetal AC signal is above the threshold intensity value.

Additionally, or alternatively, systems, processes, and/or methods for generating a modeled composite DC signal are disclosed. The modeled composite DC signal may model, or correspond to, a modeled detected optical signal (or an analog or digital representation of the optical signal) incident upon an abdomen of a pregnant mammal and a fetus contained therein that has emanated from the pregnant mammal's abdomen and been detected by a photodetector. A measured composite DC signal may also be received from a photodetector communicatively coupled to the processor. The composite DC signal may correspond to an optical signal incident upon an abdomen of a pregnant mammal and a fetus contained therein that has emanated from the pregnant mammal's abdomen and, in some instances, the fetus and been detected by the photodetector;

The modeled composite DC signal and the measured composite DC signal may then be compared with one another to determine an indication of fetal hemoglobin oxygen saturation level. A result of the comparison and/or the indication may be provided to a user. In some instances, an indication of a fetal AC signal may be received prior to receipt of the composite DC signal, and it may be determined whether an intensity of the fetal AC signal is above a threshold intensity value, wherein analysis of the composite DC signal is responsive to a determination that the intensity of the fetal AC signal is above the threshold intensity value.

### BRIEF DESCRIPTION OF THE FIGURES

The present invention is illustrated by way of example, and not limitation, in the figures of the accompanying drawings in which:

FIG. 1A is a block diagram illustrating an exemplary system for determining a level of oxygen saturation for fetal hemoglobin and/or whether meconium is present in the amniotic fluid of a pregnant mammal, consistent with some embodiments of the present invention;

FIG. 1B is a block diagram of an exemplary processor-based system that may store data and/or execute instructions for the processes disclosed herein, consistent with some embodiments of the present invention;

FIG. 2A is a block diagram illustrating an exemplary fetal oximetry probe, consistent with some embodiments of the present invention;

FIG. 2B is a block diagram illustrating another exemplary fetal oximetry probe, consistent with some embodiments of the present invention;

FIG. 2C is a block diagram illustrating an exemplary fetal oximetry probe positioned within a housing, consistent with some embodiments of the present invention;

FIG. 3A provides an illustration of exemplary dimensions for layers of tissue within two different maternal abdomens with their respective fetuses, consistent with some embodiments of the present invention;

FIG. 3B provides an illustration of exemplary dimensions for layers of tissue within two different maternal abdomens with their respective fetuses, consistent with some embodiments of the present invention;

FIG. 3C provides a midsagittal plane view of pregnant mammal's abdomen with fetal oximetry probe positioned thereon, consistent with some embodiments of the present invention;

FIG. 4A illustrates an exemplary fetal oximetry probe in contact with a pregnant mammal's abdomen showing the different layers of maternal abdominal tissue, consistent with some embodiments of the present invention;

FIG. 4B illustrates another exemplary fetal oximetry probe in contact with a pregnant mammal's abdomen, consistent with some embodiments of the present invention; and

FIG. 5 provides a flowchart illustrating a process for determining an indication of a fetal hemoglobin oxygen saturation level, consistent with some embodiments of the present invention;

FIG. 6A provides a first graph of a first linear regression of photodetector readings, in volts, that correspond to DC hemoglobin oxygen saturation levels measured over time in seconds, consistent with some embodiments of the present invention;

FIG. 6B provides a second graph of a second linear regression of photodetector readings, in volts, that correspond to DC hemoglobin oxygen saturation levels measured over time in seconds, consistent with some embodiments of the present invention; and

FIG. 7 provides a flowchart illustrating another process determining an indication of a fetal hemoglobin oxygen saturation level, consistent with some embodiments of the present invention; and

FIG. 8 provides a flowchart illustrating another process, consistent with some embodiments of the present invention.

Throughout the drawings, the same reference numerals and characters, unless otherwise stated, are used to denote like features, elements, components, or portions of the illustrated embodiments. Moreover, while the subject invention will now be described in detail with reference to the drawings, the description is done in connection with the illustrative embodiments. It is intended that changes and modifications can be made to the described embodiments without departing from the true scope and spirit of the subject invention as defined by the appended claims.

### Written Description

As described herein, light of at least two separate wavelengths and/or frequencies (e.g., red, infrared, near-infrared, etc.) from a light source (e.g., a LED or laser) may be directed into the pregnant mammal's abdomen and the fetus. This light may reflect and/or backscatter from maternal and/or fetal tissue and be incident on a photodetector that may measure the detected light and convert the detected light into a voltage signal and/or digital signal that may be separated into a composite (maternal and fetal) time-varying, or pulsatile, component, which is sometimes referred to in the art as the “AC component” or “AC signal” and a composite (maternal and fetal) steady (non-pulsatile/non-time varying) component, which is sometimes referred to in the art as a “DC component” and/or “DC signal.” The composite DC signal and/or a portion of the composite DC signal contributed by the fetus may be further analyzed to determine a relative and/or absolute fetal hemoglobin oxygenation saturation for venous, or non-pulsatile, blood of the fetus, which may be used as an indicator of fetal health and/or distress.

Analysis of a transabdominally-obtained composite non-pulsatile, or DC signal, for a pregnant mammal and her fetus may provide an indication of a hemoglobin oxygen saturation level for the fetus' non-pulsatile, or venous, blood, which may be an indicator for fetal distress such as fetal acidosis. The analysis of composite DC signals allows for the determination of a relative and/or absolute indication of fetal hemoglobin saturation level for non-pulsatile, or venous, blood without requiring processing of composite AC and/or DC signals to, for example, isolate a fetal portion of the composite DC signal and/or a composite AC and DC signal analysis of a fetal pulsatile, or AC, signal thereby reducing processing complexity and resources required to ascertain fetal hemoglobin oxygen saturation levels via more traditional means.

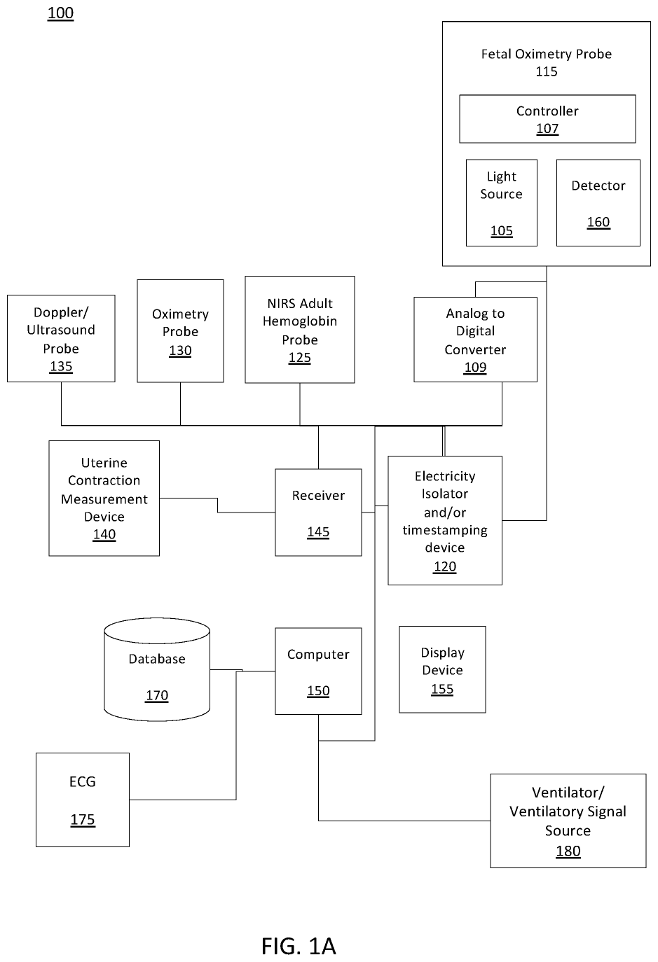

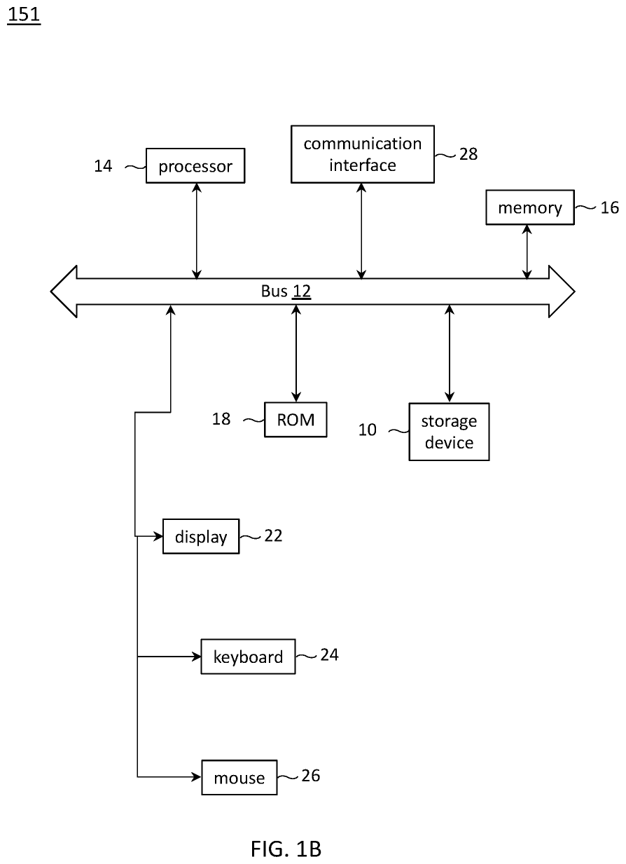

FIG. 1A provides an exemplary system 100 for detecting and/or determining fetal hemoglobin oxygen saturation levels. The components of system 100 may be coupled together via wired and/or wireless communication links. In some instances, wireless communication of one or more components of system 100 may be enabled using short-range wireless communication protocols designed to communicate over relatively short distances (e.g., BLUETOOTH®, near field communication (NFC), radio-frequency identification (RFID), and Wi-Fi) with, for example, a computer or personal electronic device (e.g., tablet computer or smart phone) as described below.

System 100 includes a light source 105, a controller 107, and a detector 160 that, at times, may be housed in a single housing, which may be referred to as fetal oximetry probe 115. Controller 107 may be configured to control an operation (e.g., on/off, intensity of light emitted by light source 105, filtering of a signal detected by detector 160, etc.) of light source 105 and/or detector 160. Light source 105 may include a single, or multiple light sources and detector 160 may include a single, or multiple detectors. In some embodiments, system 100 may include an analog to digital converter 109 configured to convert an analog signal (e.g., voltage) provided by detector 160 into a digital signal. In some embodiments, analog to digital converter 109 may be housed in fetal oximetry probe 115 as, for example, a component of controller 107. Alternatively, analog to digital converter 109 may be a component included in and/or coupled to computer 150.

Light sources 105 may transmit light at light of one or more wavelengths, including NIR, into the pregnant mammal's abdomen. Light sources 105 may be, for example, a LED, and/or a LASER, a tunable light bulb and/or a tunable LED that may be coupled to a fiber optic cable. On some occasions, the light sources may be one or more fiber optic cables optically coupled to a laser and arranged in an array. In some instances, the light sources 105 may be tunable or otherwise user configurable while, in other instances, one or more of the light sources may be configured to emit light within a pre-defined range of wavelengths. Additionally, or alternatively, one or more filters (not shown). These filters/polarizers may also be tunable or user configurable.

An exemplary light source 105 may have a relatively small form factor and may operate with high efficiency, which may serve to, for example, conserve space and/or limit heat emitted by the light source 105. In one embodiment, light source 105 is configured to emit light in the range of 770-850 nm. In some examples, light source 105 may be configured so that it does not emit light that may, for example, irritate or burn the skin of the patient and/or harm the fetus. This may be achieved by, for example, configuring and/or instructing light source 105 to emit a high-intensity/high-power pulse of light for a short time duration. This high-intensity/high-power pulse of light may be used to, for example, improve a likelihood that detectors like detector 160 positioned relatively far away from the light source will receive sufficient light to detect following the light's transmission into the pregnant mammal's abdomen and emission therefrom in a manner that does not harm the pregnant mammal or her fetus. Additionally, or alternatively, one or more light source(s) 105 may be configured to emit light in a time division multiplexed manner so that, for example, signals received from each of a plurality of detectors, like detector 160, may be distinguished from one another. Light emitted in a time division multiplexed manner may be utilized for detectors that are relatively close to the light source(s) 105.

Detector 160 may be configured to detect a light signal emitted from the pregnant mammal and/or the fetus via, for example, transmission and/or back scattering. Detector 160 may convert this light signal into an electronic signal, which may be communicated to a computer or processor and/or an on-board transceiver that may be capable of communicating the signal to the computer/processor. This emitted light might then be processed in order to determine how much light, at various wavelengths, passes through the fetus and/or is reflected and/or absorbed by the fetal oxyhemoglobin and/or de-oxyhemoglobin so that a fetal hemoglobin oxygen saturation level may be determined. This processing will be discussed in greater detail below.

Exemplary detectors include, but are not limited to, cameras, traditional photomultiplier tubes (PMTs), silicon PMTs, avalanche photodiodes, and silicon photodiodes. In some embodiments, the detectors will have a relatively low cost (e.g., $50 or below), a low voltage requirement (e.g., less than 100 volts), and non-glass (e.g., plastic) form factor. In other embodiments, (e.g., contactless pulse oximetry) a sensitive camera may be deployed to receive light emitted by the pregnant mammal's abdomen. For example, detector 160 may be a sensitive camera adapted to capture small changes in fetal skin tone caused by changes in cardiovascular pressure associated with fetal myocardial contractions. In these embodiments, detector 160 and/or fetal oximetry probe 115 may be in contact with the pregnant mammal's abdomen, or not, as this embodiment may be used to perform so-called contactless pulse oximetry. In these embodiments, light sources 105 may be adapted to provide light (e.g., in the visible spectrum, near-infrared, etc.) directed toward the pregnant mammal's abdomen so that the detector 160 is able to receive/detect light emitted by the pregnant mammal's abdomen and fetus. The emitted light captured by detector 160 may be communicated to computer 150 for processing to convert the images to a measurement of fetal hemoglobin oxygen saturation according to, for example, one or more of the processes described herein.

A fetal oximetry probe 115, light source 105, and/or detector 160 may be of any appropriate size and, in some circumstances, may be sized to accommodate the size of the pregnant mammal using any appropriate sizing system (e.g., waist size and/or small, medium, large, etc.). Exemplary lengths for a fetal oximetry probe 115 include a length of 4 cm-40 cm and a width of 2 cm-10 cm. In some circumstances, the size and/or configuration of a fetal oximetry probe 115, or components thereof, may be responsive to skin pigmentation of the pregnant mammal and/or fetus. In some instances, the fetal oximetry probe 115 may be applied to the pregnant mammal's skin via tape or a strap that cooperates with a mechanism (e.g., snap, loop, etc.) (not shown). In some embodiments, fetal oximetry probe 115 may be configured as a multiparameter unit that may be configured to, for example, communicate both ways with, for example, computer 150 and/or a processor to, for example, integrate, share, and/or store data amongst the different components of system 100.

In some embodiments, fetal oximetry probe 115 and/or a component thereof, such as controller 107, may be associated with a counter that limits a number of, for example, on/off cycles fetal oximetry probe 115 may cycle through and/or limit a duration of time the fetal oximetry probe 115 may be used so that, for example, after a time limit has expired, fetal oximetry probe 115 may issue an error message or stop working/taking measurements. Limiting a lifetime of use for fetal oximetry probe 115 and/or components thereof may serve to, for example, ensure that components do not wear out or become inaccurate in their measurements.

System 100 includes a number of optional independent sensors/probes designed to monitor various aspects of maternal and/or fetal health and may be in contact with a pregnant mammal. These probes/sensors are a NIRS adult hemoglobin probe 125, an oximetry probe 130, a Doppler and/or ultrasound probe 135, and a uterine contraction measurement device 140. Not all embodiments of system 100 will include all these components. In some embodiments, system 100 may also include an electrocardiography (ECG) machine (not shown) that may be used to determine the pregnant mammal's and/or fetus' heart rate and/or an intrauterine pulse oximetry probe (not shown) that may be used to determine the fetus' heart rate. The Doppler and/or ultrasound probe 135 may be configured to be placed on the abdomen of the pregnant mammal and may be of a size and shape that approximates a silver U.S. dollar coin and may provide information regarding fetal position, orientation, and/or heart rate. In some embodiments, oximetry probe 130 may be a conventional pulse oximetry probe placed on pregnant mammal's hand, earlobe, and/or finger to measure the pregnant mammal's hemoglobin oxygen saturation. NIRS adult hemoglobin probe 125 may be placed on, for example, the pregnant mammal's 2nd finger and may be configured to, for example, use near infrared spectroscopy to calculate the ratio of adult oxyhemoglobin to adult de-oxyhemoglobin. NIRS adult hemoglobin probe 125 may also be used to determine the pregnant mammal's heart rate.

Optionally, system 100 may include a uterine contraction measurement device 140 configured to measure the strength and/or timing of the pregnant mammal's uterine contractions. In some embodiments, uterine contractions will be measured by uterine contraction measurement device 140 as a function of pressure (e.g., measured in e.g., mmHg) over time. In some instances, the uterine contraction measurement device 140 is and/or includes a tocotransducer, which is an instrument that includes a pressure-sensing area that detects changes in the abdominal contour to measure uterine activity and, in this way, monitors frequency and duration of contractions.

In another embodiment, uterine contraction measurement device 140 may be configured to pass an electrical current through the pregnant mammal and measure changes in the electrical impedance as the uterus contracts. Additionally, or alternatively, uterine contractions may also be measured via near infrared spectroscopy using, for example, light received/detected by detector 160 because uterine contractions, which are muscle contractions, are oscillations of the uterine muscle between a contracted state and a relaxed state. Oxygen consumption of the uterine muscle during both of these stages is different and these differences may be detectable using NIRS.

Measurements and/or signals from NIRS adult hemoglobin probe 125, oximetry probe 130, Doppler and/or ultrasound probe 135, and/or uterine contraction measurement device 140 may be communicated to receiver 145 for communication to computer 150 and display on display device 155 and, in some instances, may be considered secondary signals. As will be discussed below, measurements provided by NIRS adult hemoglobin probe 125, oximetry probe 130, a Doppler and/or ultrasound probe 135, uterine contraction measurement device 140 may be used in conjunction with fetal oximetry probe 115 to isolate a fetal pulse signal and/or fetal heart rate from a maternal pulse signal and/or maternal heart rate. Receiver 145 may be configured to receive signals and/or data from one or more components of system 100 including, but not limited to, fetal oximetry probe 115, NIRS adult hemoglobin probe 125, oximetry probe 130, Doppler and/or ultrasound probe 135, and/or uterine contraction measurement device 140. Communication of receiver 145 with other components of system may be made using wired or wireless communication.

In some instances, one or more of NIRS adult hemoglobin probe 125, oximetry probe 130, a Doppler and/or ultrasound probe 135, uterine contraction measurement device 140 may include a dedicated display that provides the measurements to, for example, a user or medical treatment provider. It is important to note that not all these probes may be used in every instance. For example, when the pregnant mammal is using fetal oximetry probe 115 in a setting outside of a hospital or treatment facility (e.g., at home or work) then, some of the probes (e.g., NIRS adult hemoglobin probe 125, oximetry probe 130, a Doppler and/or ultrasound probe 135, uterine contraction measurement device 140) of system 100 may not be used.

In some instances, receiver 145 may be configured to process or pre-process received signals to, for example, make the signals compatible with computer 150 (e.g., convert an optical signal to an electrical signal), amplify a received signal, and/or improve signal to noise ratio (SNR) by, for example, performing Fast Fourier transforms (FFT), bandwidth narrowing, and/or phase correlation filtering. In some instances, receiver 145 may be resident within and/or a component of computer 150. In some embodiments, computer 150 may amplify or otherwise condition the received detected signal to, for example, improve the signal-to-noise ratio.

Receiver 145 may communicate received, pre-processed, and/or processed signals to computer 150. Computer 150 may act to process the received signals, as discussed in greater detail below, and facilitate provision of the results to a display device 155. Exemplary computers 150 include desktop and laptop computers, servers, tablet computers, personal electronic devices, mobile devices (e.g., smart phones), Internet of things (IoT) that may enable remote patient/pregnant mammal monitoring, and the like. Exemplary display devices 155 are computer monitors, tablet computer devices, and displays provided by one or more of the components of system 100. In some instances, display device 155 may be resident in receiver 145 and/or computer 150. Computer 150 may be communicatively coupled to database 170, which may be configured to store information regarding physiological characteristic and/or combinations of physiological characteristic of pregnant mammals and/or their fetuses, impacts of physiological characteristic on light behavior, information regarding the calculation of hemoglobin oxygen saturation levels, calibration factors, and so on. In some embodiments, database 170 may be local (e.g., coupled to computer 150) and/or remote (e.g., a cloud-computing database). In some embodiments, computer 150 may be configured and/or programmed to run one or more modeling and artificial intelligence programs using one or more sets of instructions (e.g., instructions for execution of one or more processes and/or process steps disclosed herein) stored on, for example, a memory of the computer and/or an external memory. At times, computer 150 may be communicatively coupled to external computing resources (e.g., a cloud computing environment and/or machine learning architecture) that may, in some instances, employed to generate, for example, modeled composite and/or fetal DC signals as described herein.

In some embodiments, a pregnant mammal may be electrically insulated from one or more components of system 100 by, for example, an electricity isolator and/or timestamping device 120. Exemplary electricity insulators and/or timestamping device 120 include switches, circuit breakers, ground fault switches, and fuses.

System 100 may also include an electrocardiography (ECG) machine 175, and/or a ventilatory/respiratory signal source 180. ECG 175 may be used to determine the pregnant mammal's and/or fetus's heart rate. In some embodiments, ECG 175 may be a fetal ECG that is used internally via, for example, placement in the birth canal and may be used to determine the fetus's heart rate.

In some embodiments, system 100 may include a ventilatory/respiratory signal source 180 that may be configured to monitor the pregnant mammal's respiratory rate and provide a respiratory signal indicating the pregnant mammal's respiratory rate to, for example, computer 150. Additionally, or alternatively, ventilatory/respiratory signal source 180 may be a source of a ventilatory signal obtained via, for example, cooperation with a ventilation machine. Exemplary ventilatory/respiratory signal sources180 include, but are not limited to, a carbon dioxide measurement device, a stethoscope and/or electronic acoustic stethoscope, a device that measures chest excursion for the pregnant mammal, and a pulse oximeter. A signal from a pulse oximeter may be analyzed to determine variations in the PPG signal that may correspond to respiration for the pregnant mammal. Additionally, or alternatively, ventilatory/respiratory signal source 180 may provide a respiratory signal that corresponds to a frequency with which gas (e.g., air, anesthetic, etc.) is provided to the pregnant mammal during, for example, a surgical procedure. This respiratory signal may be used to, for example, determine a frequency of respiration for the pregnant mammal.

In some embodiments, measurements provided by NIRS adult hemoglobin probe 125, oximetry probe 130, a Doppler and/or ultrasound probe 135, uterine contraction measurement device 140, ECG 175, and/or ventilatory/respiratory signal source 180 may be used in conjunction with fetal oximetry probe 115 to isolate a fetal pulse signal and/or fetal heart rate from a maternal pulse signal and/or maternal heart rate.

FIG. 1B provides an example of a processor-based system 151 that may be configured and/or programmed to store and/or execute instructions for the processes described herein. Processor-based system 151 may be representative of, for example, computing device 150. Note, not all the various processor-based systems which may be employed in accordance with embodiments of the present invention have all the features of system 151. For example, certain processor-based systems may not include a display inasmuch as the display function may be provided by a client computer communicatively coupled to the processor-based system or a display function may be unnecessary. Such details are not critical to the present invention.

System 151 includes a bus 12 or other communication mechanism for communicating information, and a processor 14 coupled with the bus 12 for processing information. System 151 also includes a main memory 16, such as a random-access memory (RAM) or other dynamic storage device, coupled to the bus 12 for storing information and instructions to be executed by processor 14. Main memory 16 also may be used for storing temporary variables or other intermediate information during execution of instructions to be executed by processor 14. System 151 further includes a read only memory (ROM) 18 or other static storage device coupled to the bus 12 for storing static information and instructions for the processor 14. A storage device 10, which may be one or more of a hard disk, flash memory-based storage medium, a magnetic storage medium, an optical storage medium (e.g., a Blu-ray disk, a digital versatile disk (DVD)-ROM), or any other storage medium from which processor 14 can read, is provided and coupled to the bus 12 for storing information and instructions (e.g., operating systems, applications programs and the like).

System 151 may be coupled via the bus 12 to a display 22, such as a flat panel display, for displaying information to a user. An input device 24, such as a keyboard including alphanumeric and other keys, may be coupled to the bus 12 for communicating information and command selections to the processor 14. Another type of user input device is cursor control device 26, such as a mouse, a trackball, or cursor direction keys for communicating direction information and command selections to processor 14 and for controlling cursor movement on the display 22. Other user interface devices, such as microphones, speakers, etc. are not shown in detail but may be involved with the receipt of user input and/or presentation of output.

The processes referred to herein may be implemented by processor 14 executing appropriate sequences of processor-readable instructions stored in main memory 16. Such instructions may be read into main memory 16 from another processor-readable medium, such as storage device 10, and execution of the sequences of instructions contained in the main memory 16 causes the processor 14 to perform the associated actions. In alternative embodiments, hard-wired circuitry or firmware-controlled processing units (e.g., field programmable gate arrays) may be used in place of or in combination with processor 14 and its associated computer software instructions to implement the invention. The processor-readable instructions may be rendered in any computer language. In some embodiments, processor 14 may include and/or be communicatively coupled to controller 107 of fetal oximetry probe 115.

System 151 may also include a communication interface 28 coupled to the bus 12. Communication interface 28 may provide a two-way data communication channel with a computer network, which provides connectivity to the plasma processing systems discussed above. For example, communication interface 28 may be a local area network (LAN) card to provide a data communication connection to a compatible LAN, which itself is communicatively coupled to other computer systems. The precise details of such communication paths are not critical to the present invention. What is important is that system 151 can send and receive messages and data through the communication interface 28 and in that way communicate with other controllers, etc.

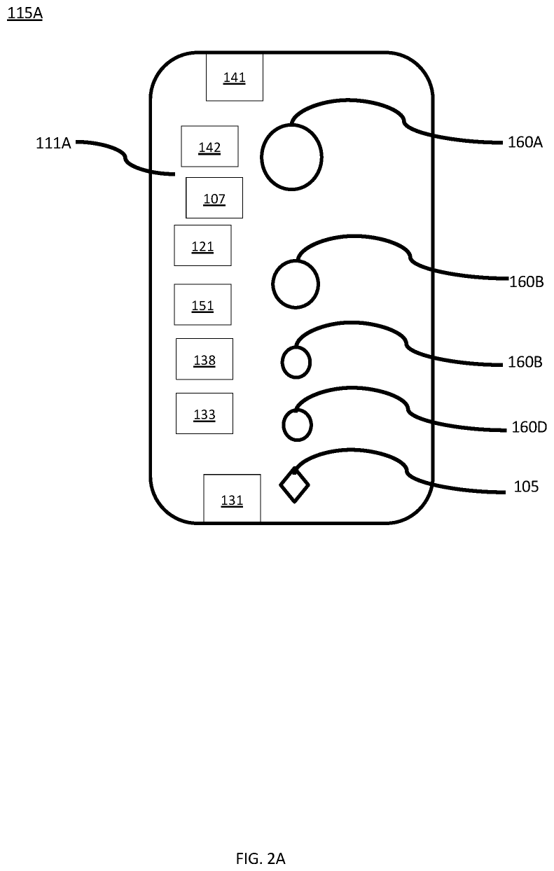

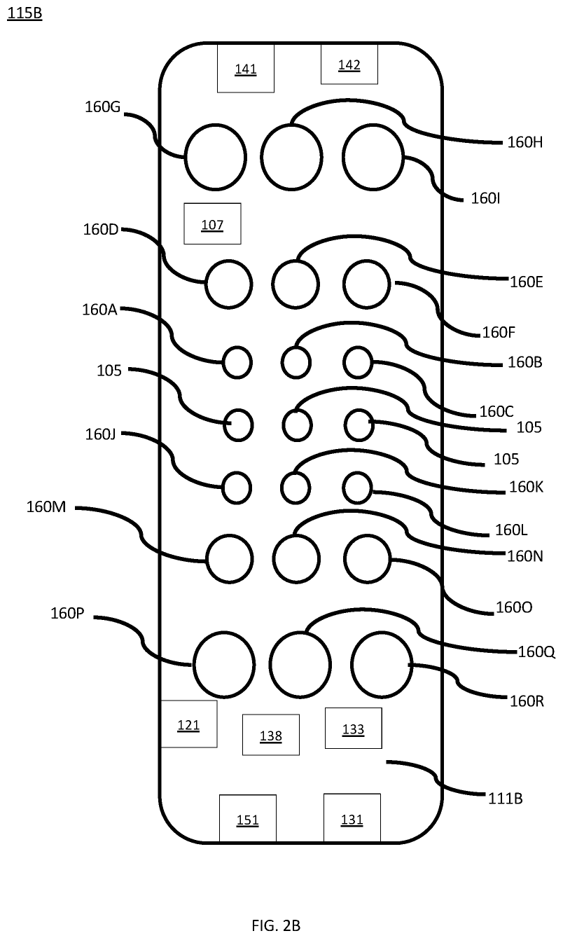

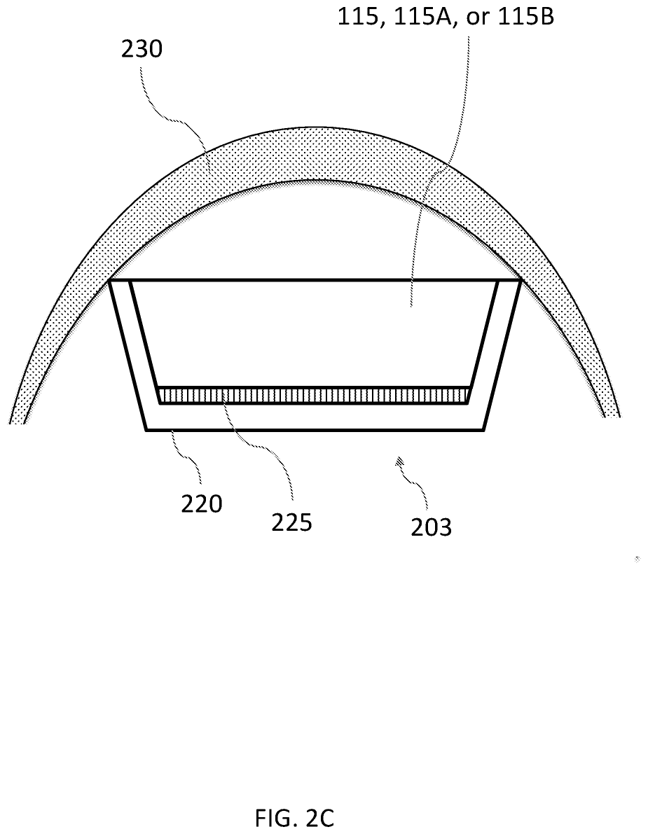

FIG. 2A is a block diagram illustrating an exemplary fetal oximetry probe 115A with housing 111A that houses a light source 105 and a plurality of detectors 160A-160D arranged in an exemplary array. Housing 111A may be any housing configured to house components of fetal oximetry probe 115A including light sources 105, the plurality of detectors 160A-160D, an optional power source 121 (e.g., a battery), a fetal depth probe 138, a maternal probe 133, a communication device (e.g., antenna or transceiver) 142, a processor 151, a power port 141, and/or a communication port 131. Exemplary fetal oximetry probe 115A includes a light source 105 substantially aligned with along the Y-axis with four detectors 160A-160D. In some embodiments, the gain, or sensitivity, of a detector 160A-160D may vary with its position relative to light source 105 so that, for example, detectors positioned further away from light source 105 (e.g., detectors 160A and 160B) have a greater gain/sensitivity than detectors positioned closer to light source 105 (e.g., detectors 160C and 160D).

In one example, fetal oximetry probe 115A may include a light source 105 configured to emit light of a plurality of wavelengths such as 735 nm, 760 nm, 810 nm, 808 nm, and/or 850 nm and each of detectors 160A-160D may be configured to detect light/photons of each of these wavelengths. An exemplary distance between light source 105 and detector 160A is 3 cm, an exemplary distance between light source 105 and detector 160B is 5 cm, an exemplary distance between light source 105 and detector 160C is 7 cm, and an exemplary distance between light source 105 and detector 160D is 10 cm.

FIG. 2B is a block diagram illustrating an exemplary fetal oximetry probe 115B with a plurality of light sources 105 and detectors 160A-160U arranged in an exemplary array within a housing 111B. Housing 111B may be any housing configured to house components of fetal oximetry probe 115B including the plurality of light sources 105, the plurality of detectors 160A-160U, optional power source 121 (e.g., a battery), fetal depth probe 138, maternal pulse oximetry probe 133, communication device (e.g., antenna or transceiver) 142, processor 151, power port 141, and/or communication port 131. Exemplary fetal oximetry probe 115A includes a light source 105 substantially aligned with along the Y-axis with four detectors 160A-160D.

Exemplary fetal oximetry probe 115B includes a row of three light sources 105 positioned in the approximate center, along the Y-axis, of housing 111B. The plurality of light sources 105 may be substantially aligned with one another along the X-axis. Housing may further include nine detectors 160A-1601 positioned above the light sources 105 in three rows with three columns and nine detectors 160K-160R positioned below the light sources 105 in three rows with three columns each. In some embodiments, the gain, or sensitivity, of a detector 160E-160R may vary with its position relative to a light source 105 so that, for example, detectors positioned further away from light source 105 have a greater gain/sensitivity as explained above with regard to fetal oximetry probe 115A.

The arrangement sources and detectors of FIGS. 2A and 2B are provided by way of example only and is not intended to limit an arrangement and/or number of light sources 105 and/or detectors 160 that may be used. Any arrangement thereof may be used to detect optical signals and convert them into the detected signal(s) discussed herein.

FIG. 2C is a block diagram illustrating an assembly 203 of an exemplary fetal oximetry probe 115, 115A, or 115B (collectively referred to below as fetal oximetry probe 115), positioned within a housing 220 that includes an optical film 225. In some embodiments, fetal oximetry probe 115 may be removably positioned within housing 220 so that it may be, for example, re-used and/or repositioned. Optionally, in some embodiments, fetal oximetry probe 115 and/or assembly 203 may be covered and/or draped by an opaque cover 230 after application to a pregnant mammal's abdomen in order to, for example, block ambient light from being detected by a detector, such as detector 160. Opaque cover 230 may also serve to protect assembly 203 from interference from other tools or equipment and/or movement by the pregnant mammal and/or individuals proximate to the pregnant mammal. Optical film 225 may be any film with optical properties that assist with detecting photons/light emanating from the pregnant mammal's abdomen.

In some cases, optical film 225 may have optical properties that are responsive to an amount of melanin in the skin of the pregnant mammal and/or fetus and, at times, may be melanin tinted. In some cases, optical film 225 may be a gel that is applied to housing 220 prior to insertion of fetal oximetry probe therein and/or an adhesive layer that attaches housing 220 and/or fetal oximetry probe 115 directly to the pregnant mammal's abdomen. Additionally, or alternatively, an optical film and/or gel may be applied to the maternal abdomen prior to placement of housing 220 and/or fetal oximetry probe 115 thereon. At times, the optical film and/or gel may be configured to include an optical absorber signature that may be detected by a detector 160 and/or understood by a processor receiving a signal from the detector. Additionally, or alternatively, the optical film and/or gel may be encoded with information regarding, for example, a patient identifier, a device identifier, an identifier for the gel, etc. An optical absorber signature and/or encoding of the optical film and/or gel may be achieved by, for example, inclusion of, for example, a nominal absorber, quantum dots, a non-decaying fluoresce dye, and/or a decaying fluoresce dye. In some cases, optical film 225 may have sufficient optical density (e.g., a tint that is black or grey) to reduce an optical short-circuit path, which may also be referred to as a shunt path and may thereby reduce effects of a short shunting light path between a light source and detector of fetal oximetry probe 115.

In some embodiments, housing 220, fetal oximetry probe 115, and/or assembly 203 may include (via, for example, an optical code, a digital chip or RFID tag) and/or be associated with information that provides an identifier of the housing 220, fetal oximetry probe 115, and/or assembly 203. The identifier may be associated with various parameters (e.g., calibration compensation, manufacturing information, wavelengths of light used, optical film characteristics, etc.) regarding an operation of assembly 203 or components thereof.

In some instances, an optical signature of optical film 225, an optical gel and/or an optical adhesive may serve to validate that a detected composite signal received by a detector like detector 160 is compliant with one or more requirements (e.g., signal-to-noise ratio, signal strength, etc.). For example, an absence of an expected optical signature in a detected composite signal may serve to indicate that a needed and/or desired optical film/gel/adhesive has not been used when taking transabdominal fetal oximetry measurements.

Additionally, or alternatively, analysis of a signature (e.g., ratio optical signals) of a detected composite signal signature measured by fetal oximetry probe 115 when an optical film/gel/adhesive like optical film 225 may assist with determining whether an optical shunt between the detector and the pregnant mammal's skin is, or has, occurred. One way to detect that an optical shunt has occurred is to determine if a signature of a detected composite signal aligns and/or is otherwise consistent with an expected (based on, for example, one or more properties of the optical film/gel/adhesive) signature of the detected composite signal because when shunting is present in the detected composite signal, the signature of a detected composite signal may not align with an expected signature of the detected composite signal.

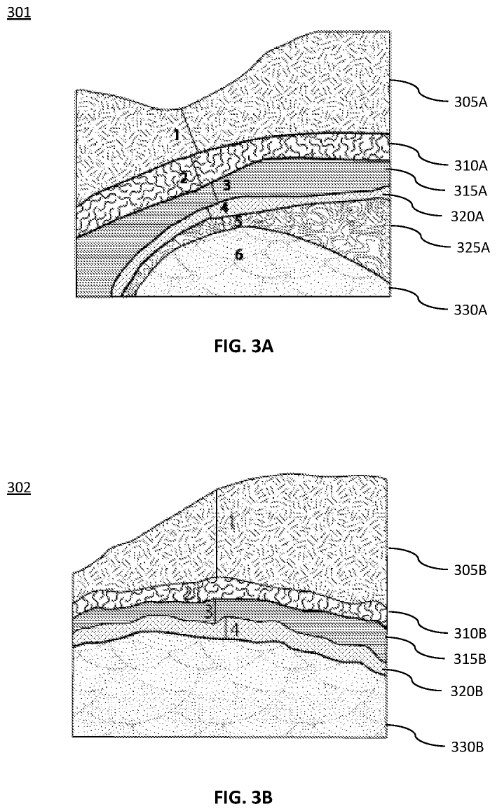

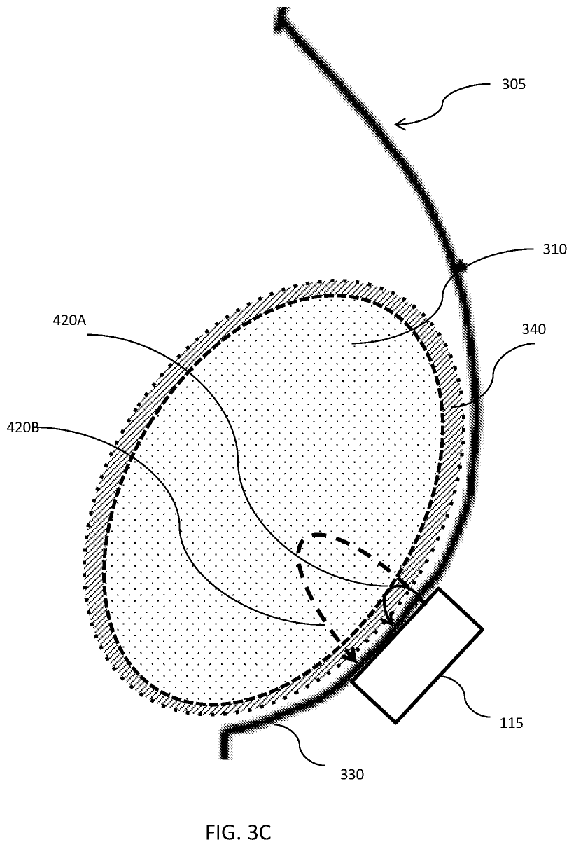

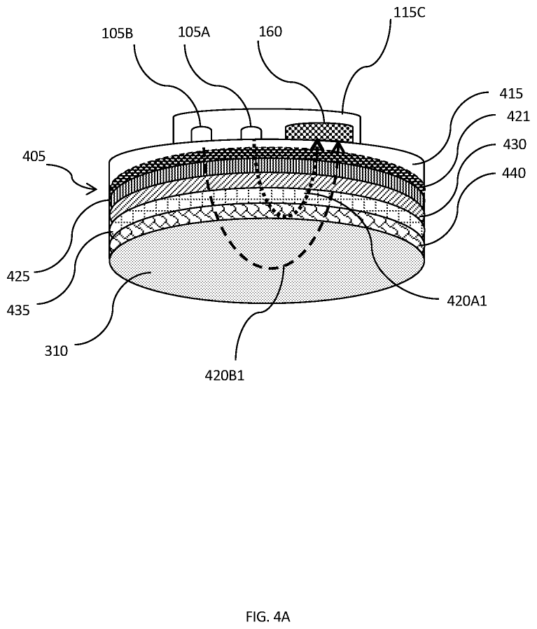

FIGS. 3A and 3B provide illustrations 301 and 302, respectively, of some layers of tissue present in two different maternal abdomens with their respective fetuses included in the illustration. Information used to generate illustrations 301 and 302 may be received from, for example, ultrasound imaging devices (e.g., Doppler/ultrasound probe 135) and/or MRI images.

Illustrations 301 and 302 provide exemplary dimensions for some layers of maternal tissue positioned proximate to a placement of a fetal oximetry probe 115 as well as the fetus including a depth of the fetus within the respective pregnant mammal's abdomen. A depth of a fetus may be understood as, for example, a distance between the epidermis of the pregnant mammal and the epidermis of the fetus and/or the aggregate width of the layers of maternal tissue and amniotic fluid. Illustration 301 shows maternal abdominal tissue for a fetus that has reached 29 weeks of gestation. The layers of tissue shown in illustration 301 include a subcutaneous fat layer 305A, an abdominal muscle (skeletal muscle) layer 310A, an intraperitoneal fat layer 315A, a uterine wall (smooth muscle) layer 320A, an amniotic fluid layer 325A, and a fetus 330A. Measurements for a width of each of these layers and are taken at a position proximate to (e.g., underneath) fetal oximetry probe 115. The approximate location for where width measurements are taken is represented by a line connecting a top and bottom of the layer of interest. For example, in FIG. 3A, a width of subcutaneous fat layer 305A is represented by line 1, a width of abdominal muscle layer 310 is represented by line 2, a width of intraperitoneal fat layer 315A is represented by line 3, a width of uterine wall layer 320A is represented by line 4, and a width of amniotic fluid layer 325A is represented by line 5. Approximate dimensions for these layers of maternal tissue that are positioned proximate to (e.g., underneath) fetal oximetry probe 115 are:
Subcutaneous fat layer 305A: 10.2 mm (represented by line 1);
Abdominal muscle layer 310A: 7.1 mm (represented by line 2);
Intraperitoneal fat layer 315A: 2.0 mm (represented by line 3);
Uterine wall layer 320A: 3.1 mm (represented by line 4);
Amniotic fluid layer 325A: 3.6 mm (represented by line 5); and
Fetus 330A. A total distance from the maternal epidermis to the epidermis of fetus 330A (i.e., fetal depth) in this example is 28 mm.

The fetus shown in illustration 302 of FIG. 3B has reached 35 weeks of gestation. The layers of tissue shown in illustration 302 include a subcutaneous fat layer 305B, an abdominal muscle (skeletal muscle) layer 310B, an intraperitoneal fat layer 315B, a uterine wall (smooth muscle) layer 320B, and a fetus 330B. Measurements for a width of each of these layers and are taken at a position proximate to (e.g., underneath) fetal oximetry probe 115. The approximate location for where width measurements are taken is represented by a line connecting a top and bottom of the layer of interest. For example, in FIG. 3B, a width of subcutaneous fat layer 305B is represented by line 1, a width of abdominal muscle layer 310 is represented by line 2, a width of intraperitoneal fat layer 315B is represented by line 3, and a width of uterine wall layer 320B is represented by line 2. Approximate dimensions for the layers of maternal tissue that are positioned proximate to (e.g., underneath) fetal oximetry probe 115 are:
Subcutaneous fat layer 305B: 11.3 mm (represented by line 1);
Abdominal muscle layer 310B: 3.1 mm (represented by line 2);
Intraperitoneal fat layer 315B: 3.1 mm (represented by line 3);
Uterine wall layer 320B: 2.3 mm (represented by line 4); and
Fetus 330B.

A total distance from maternal skin to fetus (i.e., fetal depth) in this example is 19.8 mm. Because the fetus is more developed and larger at 35 week's gestation, a width of the amniotic fluid is negligible and is not included in this example. In addition, for illustrations 301 and 302, a width of the skin of the pregnant mammal is also negligible at approximately 1-1.5 mm.

In some embodiments, the fetus 330A and/or fetal layer 330B may be divided into one or more additional layer(s) (not shown). These layers may pertain to, for example, one or more of vernix, hair, skin, bone, etc. In some embodiments, information regarding one or more of these layers (e.g., melanin content of fetal skin and/or hair color) may be deduced from, for example, parentage of the fetus, genetic testing of the fetus, and/or direct observation of the fetus via, for example, an optic scope and/or transvaginal examination.

FIG. 3C illustrates provides a midsagittal plane view of pregnant mammal's 305 abdomen with fetal oximetry probe 115 positioned thereon. As shown in FIG. 3, the pregnant mammal's abdomen 305 includes an approximation of a fetus 330, a uterus 340, and maternal tissue (e.g., skin, muscle, etc.) 330. Fetal oximetry probe 115 may be positioned anywhere on the pregnant mammal's abdomen and, in some instances, more than one fetal oximetry probe 115 may be placed on the pregnant mammal's abdomen. FIG. 3C also shows a first optical signal 420A being projected into the pregnant mammal's abdomen where the depth of penetration of first optical signal 420A is only to the edge of the uterine wall 340 and then is back scattered, or transmitted through, into a detector of fetal oximetry probe 115 like detector 160. FIG. 3C further shows a second optical signal 420B being projected into the pregnant mammal's abdomen and penetrates fetus 330 prior to being detected by detector 160. First optical signal 420A may include light of a single wavelength or a plurality of wavelengths that may be, for example, red or NIR. In some embodiments, first optical signal may include light of two distinct wavelengths or ranges of wavelengths, one red and one NIR. Second optical signal 420B may include light of a single wavelength or a plurality of wavelengths that may be, for example, red or NIR. The wavelength(s) of second optical signal 420B may be different from those of first optical signal 420A and/or projected into the pregnant mammal's abdomen at different times so that second optical signal 420B may be distinguished from first optical signal 420A during processing of detected portions of first and second optical signals 420A and 420B, respectively. In some embodiments, first and second optical signals 420A and 420B may include light of two distinct wavelengths or ranges of wavelengths, one red and one NIR that are slightly different from one another. For example, first optical signal 420A may be red and second optical signal 420B may be NIR, both first and second optical signals 420A and 420B may be red or NIR. In these examples, the wavelengths of first and second optical signals 420A and 420B may be selected so that any differences in their respective path lengths will be negligible. The two wavelengths may enable pulse oximetry calculations using, for example, differences in absorption, or (μₐ(λ)), and/or scattering (μₛ(λ)) of the optical signal using, for example, the Lambert-Beer or modified Lambert-Beer calculations as, for example, described herein.

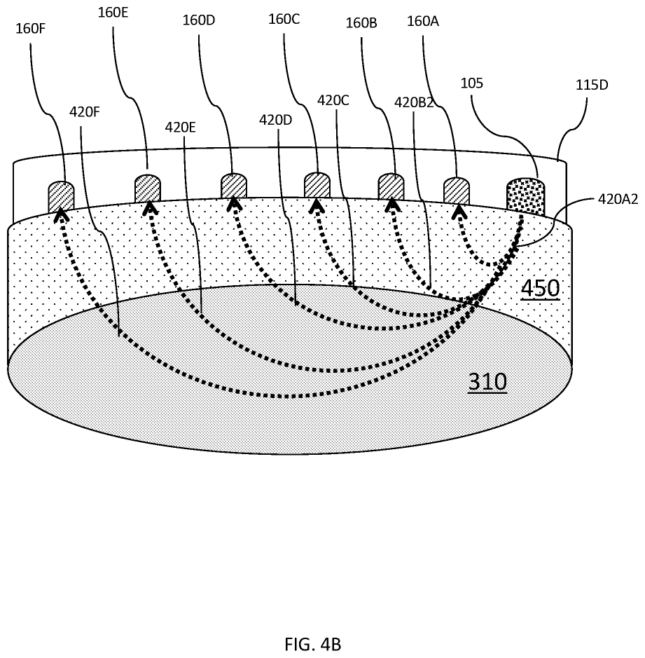

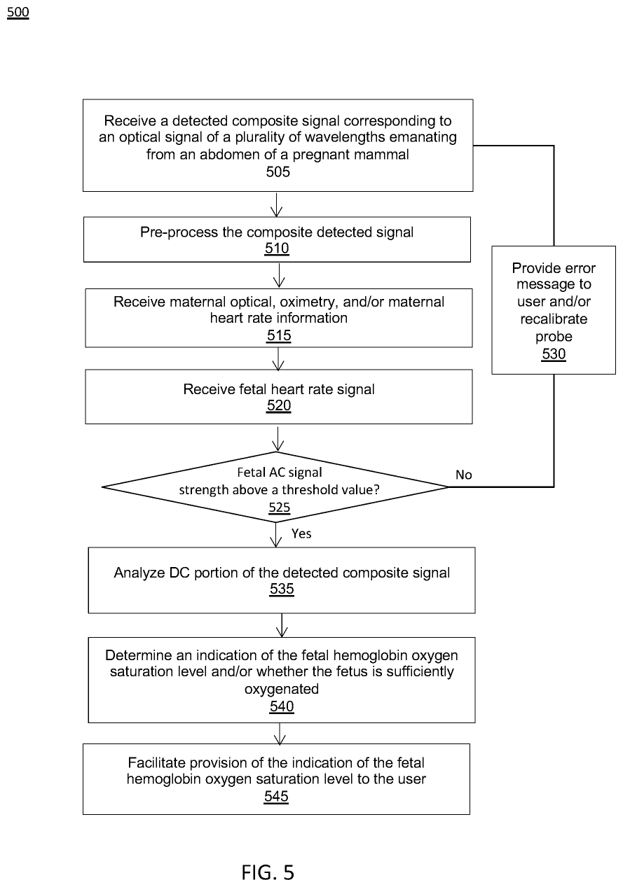

FIG. 4A illustrates an exemplary fetal oximetry probe 115C in contact with a pregnant mammal's abdomen in a manner similar to that shown in FIG. 3. FIG. 4A also shows a plurality of layers of tissue. More specifically, FIG. 4A shows a first layer that represents a maternal skin layer 415, a second layer that represents a maternal subcutaneous fat layer 421, a third layer that represents a maternal abdominal muscle (skeletal muscle) layer 425, a fourth layer that represents a maternal intraperitoneal fat layer 430, a fifth layer that represents a uterine wall (smooth muscle) layer 435, a sixth layer that represents an amniotic fluid layer 440, and a seventh layer that represents the fetus 330.

Fetal oximetry probe 115C includes a first light source 105A that emits first light beam 420A1, a second light source 105B that emits second light beam 420, and a detector 160. First and/or second light beams 420A1 and/or 420B1 may include light of a single, or multiple, wavelengths and may be within, for example, the red, NIR, or infra-red spectrum. In some circumstances, characteristics of light beam 420A1 may be different from the wavelength of light beam 420B1 and/or may be projected into the pregnant mammal's abdomen at a different time to enable distinguishing light projected from the two light sources when it is received by detector 160 and processed according to one or more of the processes described herein. In some embodiments, fetal oximetry probe 115C may include a filter (not shown) for detector 160 that may be attenuated to so that detector 160 detects and equal amount of light from first and second light sources 105A and 105B.

In many instances, a depth of light propagation through the pregnant mammal's abdomen is dependent on a distance between a light source and a detector. In some embodiments, the position of first light source 105A and/or second light source 105B may be adjusted (e.g., moved closer to, or further away from, detector 160) to, for example, adjust a depth of penetration for the light emitted therefrom. The adjustment may be facilitated by, for example, a track or other positioning device included in fetal oximetry probe 115C (not shown). In some instances, the positioning of first light source 105A and/or second light source 105B may be adjusted responsively to a depth of fetus 330 within the pregnant mammal's abdomen (i.e., a measurement of the width of maternal tissue 405 positioned between the fetal oximetry probe 115C and the fetus 330). A measurement of a depth of fetus 330 within the pregnant mammal's abdomen may be provided by, for example, an ultrasound or Doppler probe like Doppler/ultrasound probe 135 and/or an MRI image, an illustration of a portion of which is shown in illustrations 301 and 302.

In some embodiments, first light source 105A may be positioned relative to detector 160 so that light emitted from first light source (i.e., light beam 420A1) only propagates through the maternal tissue 405 and does not reach fetus 330. Second light source 105B may be positioned further away (relative to first light source 105A) from detector 160 so that light projected by second light source 105B (i.e., light beam 420B1) projects deeper into the pregnant mammal's abdomen than light beam 420A1 and back scattering therefrom and/or transmission therethrough are detected by detector 160. Stated differently, light source 105A may be positioned so light beam 420A1 only projects into maternal tissue 405 so that the portion of light beam 420A1 detected by detector 160 may only be back scattered from and/or transmitted through from maternal tissue 405 and not the fetus 330 while light source 105B may be positioned so light beam 420B1 projects into both maternal tissue 405 and fetus 330 so that the portion of light beam 420B1 detected by detector 160 may be back scattered from and/or transmitted through from maternal tissue 405 and the fetus 330. This positioning of first light source 105A may facilitate short separation (SS) measurements and the path of first light beam 420A1 and/or the detected amounts of first light beam 420A1 by detector 160 may be referred to herein as a SS channel and//or a short separation signal. This positioning of second light source 105B may facilitate long separation (LS) measurements and the path of second light beam 420B1 and/or the detected amounts of second light beam 420B1 by detector 160 may be referred to herein as a LS channel.

FIG. 4B illustrates an exemplary fetal oximetry probe 115B positioned on a pregnant mammal's abdomen. The maternal tissue of the pregnant mammal's abdomen is represented as maternal tissue 405 and a fetus within the pregnant mammal's abdomen is represented as fetus 410.

Fetal oximetry probe 115D has one light source 105 and six detectors 160A, 160B, 160C, 160D, 160E, and 160F, each of which have a different position relative to source 105 with first detector 160 A being the closest to source 105 and sixth detector 160F being the furthest away from source 105. A position of a detector 160A-160F relative to source 105 may be referred to herein as a source/detector distance. In some examples, detectors 160A-160F may be arranged linearly and may be positioned 1 cm apart from one another so that first detector 160A is positioned 1 cm away from source 105, second detector 160B is positioned 1 cm away from first detector 160A, third detector 160C is positioned 1 cm away from second detector 160B, fourth detector 160D is positioned 1 cm away from third detector 160C, fifth detector 160E is positioned 1 cm away from fourth detector 160D, and sixth detector 160F is positioned 1 cm away from fifth detector 160E.

Source 105 may project an optical signal 420 into the pregnant mammal's abdomen 405 and a resultant optical signal may be detected by one or more of detector(s) 160A-160F. It is expected that the detectors positioned closer to source 105 will detect a portion of the optical signal that has been incident on the pregnant mammal's abdomen 405 but not fetus 330 and, in some embodiments, first detector 160A and/or second detector 160B may be positioned via, for example, setting of a source/detector distance, so that a majority, if not all, of an optical signal 420A2 and 420B1 detected by first and second detectors 160A and 160B, respectively, has only been incident of the pregnant mammal's abdomen 405 (i.e., is not incident on the fetus). Third-sixth detectors 160C-160F may detect portions of the optical signal 420C, 420D, 420E, and 420F that are incident on the pregnant mammal 405 and fetus 330 as shown in FIG. 4. In some cases, third detector 160C may be positioned 3-5 cm away from the light source and sixth detector 160F may be positioned 6-10 cm away from the light source. Additionally, or alternatively, third-sixth detectors 160C-160F may be positioned within 4-10 cm of the light source.

As the source/detector distance increases a proportion of the optical signal that corresponds to light that was incident on fetus 330 increases. Thus, optical signal 420F may include a higher proportion of light that was incident on the fetus than, for example, optical signal 420E or 420D.

FIG. 5 provides a flowchart illustrating a process 500 for determining an indication of a fetal hemoglobin oxygen saturation level. Process 500 may be executed by, for example, any of the system or system components described herein.

Initially, a detected composite signal may be received from a photodetector (e.g., detector 160) and/or an analog to digital converter (e.g., analog to digital converter 109) by a processor and/or computer like computer 150 (step 505). The detected composite signal may include a maternal AC signal, a fetal AC signal, a maternal DC signal, and/or a fetal DC signal.

The detected composite signal may be received from, for example, a photodetector, a transceiver coupled to the photodetector, and/or a fetal oximetry probe such as fetal oximetry probe 115. The detected composite signal may correspond to an optical signal of a plurality of wavelengths emanating (via, for example, transmission, back scattering, and/or reflection) from the abdomen of a pregnant mammal and/or her fetus. Light incident upon, and exiting from, the pregnant mammal's abdomen may be generated by one or more light sources, like light source 105 and may be of any acceptable frequency or wavelength (e.g., near infra-red (NIR)) and/or combination of frequencies and/or wavelengths. In some embodiments (e.g., when multiple detectors are used), the received detected composite signals may include and/or be associated with a detector identifier (e.g., code) so that a position of a particular detected composite signal may be known. This location may then be used to analyze the received detected composite signals to determine various factors of the detected light and/or imaged tissue. In some embodiments, the received detected composite signal may also include and/or be associated with a time stamp inserted into and/or associated with the signal by, for example, a component of system 100 and/or 101 such as electricity isolator and/or timestamping device 120.

Optionally, in step 510, the detected composite signal may be pre-processed and/or filtered to, for example, remove noise, ambient light, and/or motion artifacts from the pregnant mammal, fetus, and/or fetal oximetry probe.

Optionally, in step 515, maternal oximetry information and/or maternal heart rate information (e.g., a pulse) may be received from, for example, an oximeter and/or a pulse oximeter like NIRS adult hemoglobin probe 125, oximetry probe 130, and/or from an ECG machine like ECG machine 175. Additionally, or alternatively, maternal optical information may be received in step 515. Maternal optical information may be and/or correspond to an optical signal that has only traversed maternal tissue such as short separation measurement and/or short separation signal (e.g., 420A1, 420A2, and/or 420B) of the pregnant mammal's AC and/or DC signal.

Optionally, in step 520, a fetal heart rate may be received from, for example, a Doppler/ultrasound probe like Doppler/ultrasound probe 135 and/or an ECG machine like ECG machine 175. The fetal heart rate may be used to analyze the detected composite signal to determine if a pulsatile (AC) signal for the fetus is present in the detected composite signal and, if so, whether the fetal AC signal is strong enough and/or above a threshold intensity value (step 525) to indicate that the probe providing the detected composite signal is properly positioned over the fetus because, for example, an imperceptible, or weak, fetal AC signal may indicate that the portion of the detected composite signal received in step 505 contributed by the fetus is insufficient to determine an accurate indication of fetal hemoglobin oxygen concentration.

In embodiments where maternal oximetry and/or heart rate information is/are received in step(s) 510 and/or 515, this information may be used to remove portions (e.g., maternal AC and/or maternal DC signals) of the detected composite signal contributed by the pregnant mammal as part of execution of step 525. The resulting portion of the detected composite signal may be a portion of the signal (e.g., fetal DC signal and/or fetal AC signal) contributed by light incident on the fetus, which may be referred to herein as a “fetal signal”. Removal of portions of the pregnant mammal's contributions to the detected composite signal may be done by, for example, synchronizing the maternal oximetry and/or maternal heart rate information with the detected composite signal in time and then subtracting a portion of the detected composite signal that corresponds to the maternal heart rate information. A resulting signal may not include the maternal AC signal, thereby isolating the fetal AC signal. The resulting signal may also include fetal and maternal DC signals. In one example, execution of step 525 may include examining the detected composite signal to find an AC signal that is synchronized with the fetal heart rate (thereby indicating that the AC signal corresponds to the fetal AC signal) and, if so, determining if such an AC signal is strong enough for further analysis according to process 500. When the fetal signal is not of sufficient strength to be analyzed, an error message may be provided to a user (e.g., clinician or nurse) (step 530) so that, for example, a probe being used to provide the detected composite signal may be moved to a different position on the maternal abdomen to, for example, obtain a stronger and/or less noisy fetal signal. Additionally, or alternatively, an error message provided in step 530 may request that the user execute a calibration or other tuning procedure for the probe. Additionally, or alternatively, execution of step 530 may be completed by the probe automatically (i.e., without user involvement) performing a recalibration or other tuning procedure (e.g., noise reduction or filtering).

In some embodiments, steps 520-530 may not be performed when, for example, a detected composite signal is likely to include light incident on the fetus. This may be the case when, for example, an ultrasound or other device is used to locate the fetus and/or a portion of the fetus (e.g., head or back) from which to obtain an oximetry measurement and this location is used to guide placement of the fetal oximetry probe on the pregnant mammal's abdomen so that light incident on the pregnant mammal's abdomen is likely to penetrate to the fetus and, consequently, the detected composite signal is likely to include light incident on the fetus.

When the fetal oximetry probe is positioned so that the detected composite signal includes light incident on the fetus and/or when the fetal signal is of sufficient strength (when step 525 is performed), a DC portion of the fetal signal may be analyzed (step 535) to determine an indication of fetal hemoglobin oxygen saturation level thereof (step 540) and an indication of the fetal hemoglobin oxygen saturation level may be provided to the user via, for example, a display device like display device 155 (step 545). In some embodiments, the indication of fetal hemoglobin oxygen saturation level may be a relative or absolute fetal hemoglobin oxygen saturation level that may be represented as a number or percentage. Additionally, or alternatively, the indication of fetal hemoglobin oxygen saturation level may be an indication of whether the fetus is sufficiently oxygenated, in distress, and/or may be dangerously deoxygenated. This indication may be, for example, a binary value (e.g., sufficient/insufficient, healthy/not healthy, or distress/no distress), a set of values (e.g., a scale of 1-5 or 1-10), and/or an activation of a warning condition (e.g., visual and/or audible alarm) when the fetal hemoglobin oxygen saturation level may fall below a threshold value.

In some embodiments, the analysis of step 535 may include determining a number of photons detected by a photodetector like detector 160 that may be included in the detected composite signal of step 505 and/or a portion of the detected composite signal that correspond to a composite of the maternal and fetal DC signals. It is known that oxygenated hemoglobin reflects more light than deoxygenated hemoglobin (which absorbs more light than oxygenated hemoglobin). Thus, if a measurement of the number of photons included in the composite of the maternal and fetal DC signals is relatively high and/or above a threshold for sufficiently oxygenated fetal hemoglobin, this may indicate that the fetus' hemoglobin is sufficiently oxygenated without having to, for example, separately analyze the fetal and/or maternal DC signals. Conversely, if a measurement of the number of photons included in the composite of the maternal and fetal DC signals is relatively low and/or below a threshold for sufficiently oxygenated fetal hemoglobin, this may indicate that the fetus' hemoglobin is not sufficiently oxygenated and may indicate that the fetus is in distress.

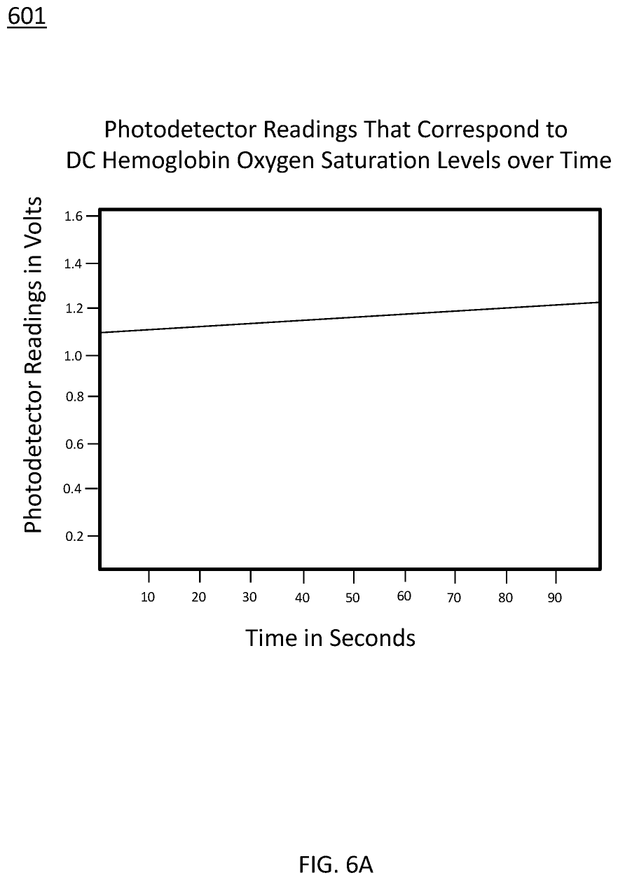

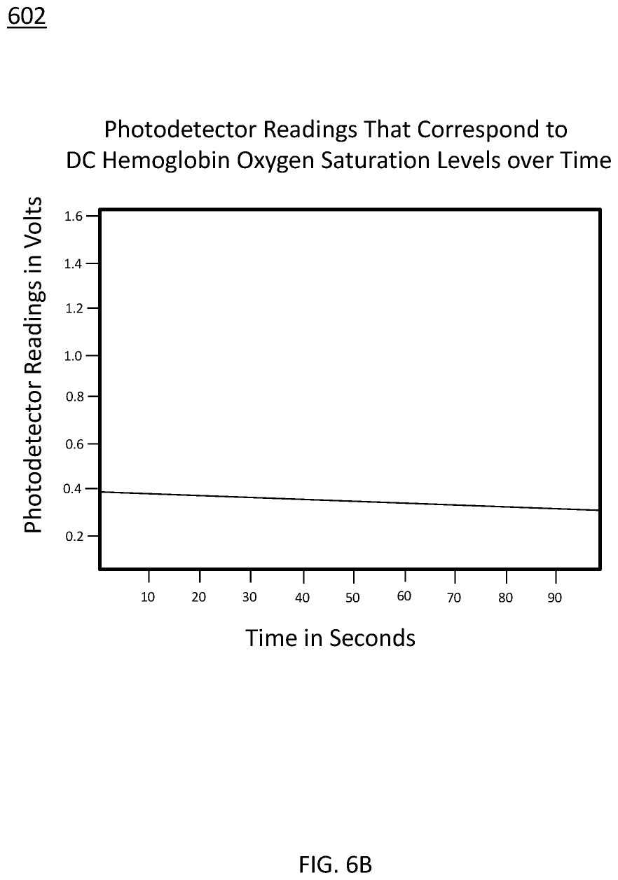

In some embodiments, measurement of a number of photons detected by a photodetector may be provided in volts and a magnitude of the voltage measurements may be proportional to an oxygen saturation level of the venous hemoglobin wherein a relatively high voltage measurement may indicate a correspondingly relatively high hemoglobin oxygen saturation level and a relatively low high voltage measurement may indicate a correspondingly relatively low hemoglobin oxygen saturation level. Exemplary voltage measurements recorded by a photodetector like detector 160 are provided by the graphs shown in FIGS. 6A and 6B, wherein FIG. 6A provides a first graph 601 showing a first linear regression, or approximation, of photodetector readings, in volts, measured by photodetector over time in seconds where values for voltage range from 1.1-1.2 volts over 100 s time and FIG. 6B provides a second graph 602 a second linear regression, or approximation, of photodetector readings, in volts, measured by photodetector over time in seconds where values for voltage range from 0.4-0.3 volts over time.

The voltage values shown in graphs 601 and 602 correspond to how many photons are present within, and/or an intensity of, an optical signal detected by the photodetector for non-pulsatile, or venous, blood (i.e., the DC signal). In some cases, the voltage measurements shown in graphs 601 and 602 may correspond to a composite DC signal of both the pregnant mammal and fetus.

In some cases, these voltage measurements may be processed (e.g., converted into a digital signal, filtered, amplified, etc.) by for example, controller 107 and/or computer 150. A resulting digital signal may then be processed and/or analyzed to calculate, or otherwise determine, a level of hemoglobin oxygenation in venous blood (i.e., DC hemoglobin oxygen saturation level) using, for example, a known (e.g., empirically determined) or calculated correspondence between voltage magnitude and/or a digital value corresponding to voltage magnitude and hemoglobin oxygen saturation levels.

The voltage values of 1.1-1.2 volts over 100 s shown in first graph 601 correspond to hemoglobin oxygen saturation levels that are sufficient to maintain fetal health and/or avoid distress (e.g., fetal oxygen desaturation and/or acidosis). This determination may be made via, for example, execution of step(s) 535 and/or 540 and may be based on, for example, a comparison of known values for the DC portion of a detected composite signal for various levels of fetal hemoglobin oxygenation, wherein a voltage between 1-1.3 is known to indicate the fetus' hemoglobin is sufficiently oxygenated (i.e., is reflecting a higher percentage of light than if the fetus' blood was not sufficiently oxygenated (i.e., deoxygenated)). Additionally, or alternatively, a determination of whether the fetus is sufficiently oxygenated may also be made by comparing the data of graph 601 with the data of graph 602, wherein a voltage between 0.5-0.2 volts may be known to indicate the fetus' hemoglobin is not sufficiently oxygenated.

Additionally, or alternatively, a determination that the fetal hemoglobin is sufficiently oxygenated may be made by, for example, comparing DC fetal hemoglobin oxygenation values calculated using the data used to generate in graph 601 and/or 602 with DC oximetry values for the pregnant mammal to determine any difference therebetween. Maternal DC oximetry values may be determined via, for example, a short separation measurement of the pregnant mammal's DC signal and/or a maternal DC signal measurement received from an oximeter and/or pulse oximeter (e.g., oximetry probe 130) that may be positioned on, for example, the maternal abdomen and/or elsewhere on her body (e.g., fingertip or ear lobe).

In some embodiments, the determination of step 545 may be made by comparing measurements regarding a DC portion of the detected composite signal over time to monitor trends thereof. In this way, it may be determined if a fetus' DC hemoglobin oxygenation is declining or improving over time during, for example, a labor and delivery process. In these embodiments, it may be assumed that maternal DC hemoglobin oxygen saturation values may remain relatively constant and, consequently, any changes to the composite DC hemoglobin oxygen saturation values may be contributed by the fetus. The assumption that maternal DC hemoglobin oxygen saturation values are relatively constant may, in some cases, be validated and/or confirmed via analysis of DC hemoglobin oxygen saturation values via an oximetry probe (e.g., oximetry probe 130) configured to measure maternal-only DC hemoglobin oxygen saturation values. Alternatively, maternal DC hemoglobin oxygen saturation values may be determined via the oximetry probe (e.g., oximetry probe 130) over time and compared with a composite maternal/fetal DC signal so that maternal changes in DC hemoglobin oxygen saturation values may be calculated over time and removed (e.g., filtered and/or subtracted) from the composite DC signal so that any remaining changes to the DC signal over time may be deduced to be contributed by the fetus (i.e., may indicate changes in DC fetal hemoglobin oxygen saturation values) over time.

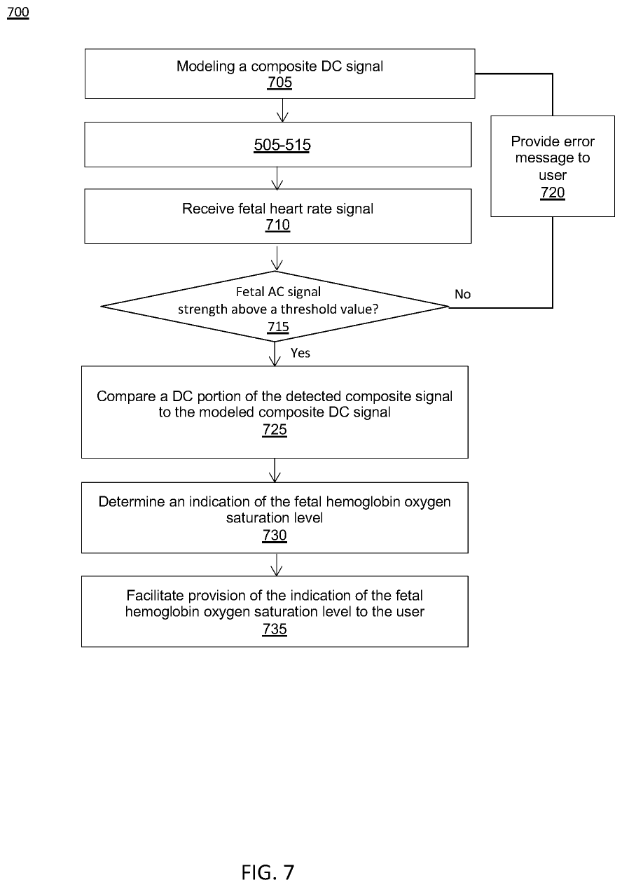

FIG. 7 provides a flowchart illustrating another process 700 for determining an indication of a fetal hemoglobin oxygen saturation level. Process 700 may be executed by, for example, any of the system or system components described herein.

Initially, a predicted value, or range of values, for a composite DC signal that indicates the fetus is sufficiently oxygenated and/or indicates that the fetus is not sufficiently oxygenated may be determined (step 705) using, for example, the maternal oximetry and/or heart rate information received in step 515. A predicted value and/or range of values for the composite DC signal may be, for example, a voltage or range of voltages for the DC portion of the detected composite signal as measured over time as shown in, for example, graphs 601 and 602 of FIGS. 6A and 6B. For example, a range of values for measurements taken by a photodetector like photodetector 160 may be between 1.0-1.3 volts may be a predicted range of values that may indicate that the fetal blood is sufficiently oxygenated and a range of values between 0.5-0.2 volts may be a predicted range of values that may indicate that the fetal blood is not sufficiently oxygenated and/or is at risk of harm.

In some embodiments, the prediction of step 705 may incorporate evaluation of, for example, one or more characteristics of, for example, the pregnant mammal, fetal depth (distance between maternal abdominal epidermis and fetal epidermis) and/or fetal oximetry probe and/or a sensitivity of a component (e.g., detector) thereof. These characteristics may be received in, for example, step 505 and/or determined in situ as part of, for example, a calibration procedure or equipment testing cycle.

Following step 705, step(s) 505, 510, and/or 515 of process 500 may be performed. Optionally, in step 710, a fetal heart rate signal may be received and in step 715, it may be determined if a fetal portion of the detected composite signal and/or fetal AC signal is of sufficient strength and/or above a threshold intensity value and, if not, an error message may be sent to a user (step 720). At times, execution of step 710 may resemble execution of step 520, execution of step 715 may resemble execution of step 525, and/or execution of step 720 may resemble execution of step 530.

In step 725, a composite (maternal and fetal) DC portion of the detected composite signal received in step 505 may be compared with the modeled composite DC signal from step 705 to determine an indication of fetal hemoglobin oxygen saturation level (step 730). In some cases, the indication of the comparison may correspond to a non-pulsatile hemoglobin oxygen saturation level that is above or below a modeled value. The indication of fetal hemoglobin oxygen saturation level may then be provided to the user (step 735). In some embodiments, execution of steps 730 and 735 may resemble execution of steps 545 and 550.

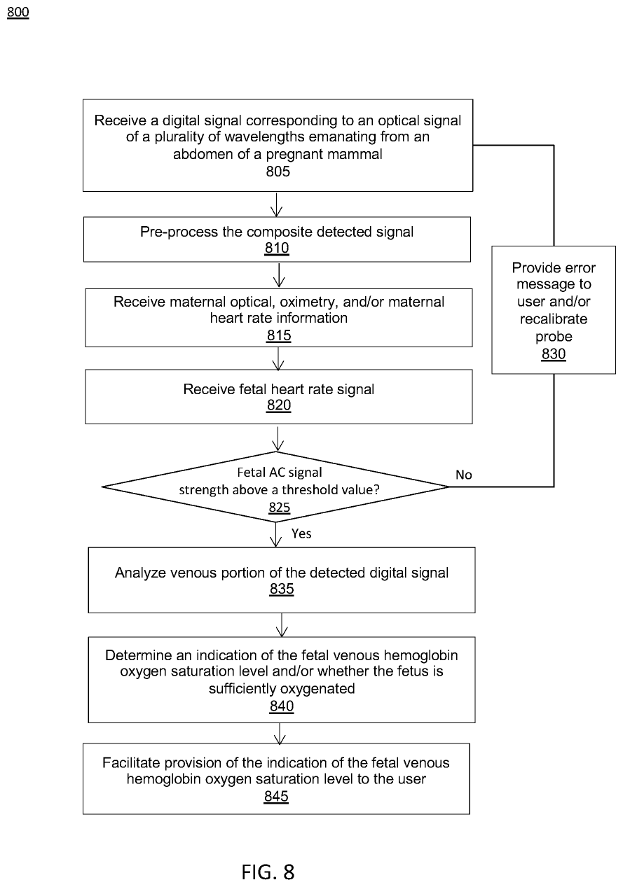

FIG. 8 provides a flowchart illustrating another process 800 for determining an indication of a fetal hemoglobin oxygen saturation level. Process 700 may be executed by, for example, any of the system or system components described herein.

Initially, a digital signal may be received from a photodetector (e.g., detector 160) and/or an analog to digital converter (e.g., analog to digital converter 109) by a processor and/or computer like computer 150 (step 805). The digital signal may include a maternal and fetal pulsatile signal and/or a maternal and fetal venous signal.

The digital signal may be received from, for example, a photodetector, a transceiver coupled to the photodetector, and/or a fetal oximetry probe such as fetal oximetry probe 115. The digital signal may correspond to an optical signal of a plurality of wavelengths emanating (via, for example, transmission, back scattering, and/or reflection) from the abdomen of a pregnant mammal and/or her fetus. Light incident upon, and exiting from, the pregnant mammal's abdomen may be generated by one or more light sources, like light source 105 and may be of any acceptable frequency or wavelength (e.g., near infra-red (NIR)) and/or combination of frequencies and/or wavelengths. In some embodiments (e.g., when multiple detectors are used), the received digital signals may include and/or be associated with a detector identifier (e.g., code) so that a position of a particular digital signal may be known. This location may then be used to analyze the received digital signals to determine various factors of the detected light and/or imaged tissue. In some embodiments, the received digital signal may also include and/or be associated with a time stamp inserted into and/or otherwise associated with the signal by, for example, a component of system 100 and/or 101 such as electricity isolator and/or timestamping device 120.

Optionally, in step 810, the digital signal may be pre-processed and/or filtered to, for example, remove noise, ambient light, and/or motion artifacts from the pregnant mammal, fetus, and/or fetal oximetry probe.

Optionally, in step 815, maternal oximetry information and/or maternal heart rate information (e.g., a pulse) may be received from, for example, an oximeter and/or a pulse oximeter like NIRS adult hemoglobin probe 125, oximetry probe 130, and/or from an ECG machine like ECG machine 175. Additionally, or alternatively, maternal optical information may be received in step 815. Maternal optical information may be and/or correspond to an optical signal that has only traversed maternal tissue such as short separation measurement and/or short separation signal of the pregnant mammal's pulsatile and/or venous signal.

Optionally, in step 820, a fetal heart rate may be received from, for example, a Doppler/ultrasound probe like Doppler/ultrasound probe 135 and/or an ECG machine like ECG machine 175. The fetal heart rate may be used to analyze the digital signal to determine if a pulsatile (AC) signal for the fetus is present in the digital signal and, if so, whether the fetal pulsatile signal is strong enough and/or above a threshold intensity value (step 825) to indicate that the probe providing the digital signal is properly positioned over the fetus because, for example, an imperceptible, or weak, fetal pulsatile signal may indicate that the portion of the digital signal received in step 805 contributed by the fetus is insufficient to determine an accurate indication of fetal hemoglobin oxygen concentration.

In embodiments where maternal oximetry and/or heart rate information is/are received in step(s) 810 and/or 815, this information may be used to remove portions (e.g., maternal pulsatile and/or maternal venous signals) of the digital signal contributed by the pregnant mammal as part of execution of step 825. The resulting portion of the digital signal may be a portion of the signal (e.g., fetal venous signal and/or fetal pulsatile signal) contributed by light incident on the fetus, which may be referred to herein as a “fetal signal”. Removal of portions of the pregnant mammal's contributions to the digital signal may be done by, for example, synchronizing the maternal oximetry and/or maternal heart rate information with the digital signal in time and then subtracting a portion of the digital signal that corresponds to the maternal heart rate information. A resulting signal may not include the maternal pulsatile signal, thereby isolating the fetal pulsatile signal. The resulting signal may also include fetal and maternal venous signals. In one example, execution of step 825 may include examining the digital signal to find a pulsatile signal that is synchronized with the fetal heart rate (thereby indicating that the pulsatile signal corresponds to the fetal pulsatile signal) and, if so, determining if such a pulsatile signal is strong enough for further analysis according to process 800. When the fetal signal is not of sufficient strength to be analyzed, an error message may be provided to a user (e.g., clinician or nurse) (step 830) so that, for example, a probe being used to provide the digital signal may be moved to a different position on the maternal abdomen to, for example, obtain a stronger and/or less noisy fetal signal. Additionally, or alternatively, an error message provided in step 830 may request that the user execute a calibration or other tuning procedure for the probe. Additionally, or alternatively, execution of step 830 may be completed by the probe automatically (i.e., without user involvement) performing a recalibration or other tuning procedure (e.g., noise reduction or filtering).

In some embodiments, steps 820-830 may not be performed when, for example, a digital signal is likely to include light incident on the fetus. This may be the case when, for example, an ultrasound or other device is used to locate the fetus and/or a portion of the fetus (e.g., head or back) from which to obtain an oximetry measurement and this location is used to guide placement of the fetal oximetry probe on the pregnant mammal's abdomen so that light incident on the pregnant mammal's abdomen is likely to penetrate to the fetus and, consequently, the digital signal is likely to include light incident on the fetus.

When the fetal oximetry probe is positioned so that the digital signal includes light incident on the fetus and/or when the fetal signal is of sufficient strength (when step 825 is performed), a venous portion of the digital signal and/or fetal signal may be analyzed (step 835) to determine an indication of fetal hemoglobin oxygen saturation level thereof (step 840) and an indication of the fetal hemoglobin oxygen saturation level may be provided to the user via, for example, a display device like display device 155 (step 845). In some embodiments, the indication of fetal hemoglobin oxygen saturation level may be a relative or absolute fetal hemoglobin oxygen saturation level that may be represented as a number or percentage. Additionally, or alternatively, the indication of fetal hemoglobin oxygen saturation level may be an indication of whether the fetus is sufficiently oxygenated, in distress, and/or may be dangerously deoxygenated. This indication may be, for example, a binary value (e.g., sufficient/insufficient, healthy/not healthy, or distress/no distress), a set of values (e.g., a scale of 1-5 or 1-10), and/or an activation of a warning condition (e.g., visual and/or audible alarm) when the fetal hemoglobin oxygen saturation level may fall below a threshold value.

In some embodiments, execution of steps 835, 840, and/or 845 may be similar to execution of steps 535, 540, and/or 545, respectively.

In some embodiments, two or more of the processes, or portions thereof, may be combined in any order and executed together.

Hence, systems, devices, and methods for determining fetal oxygen level have been herein disclosed. In some embodiments, use of the systems, devices, and methods described herein may be particularly useful during the labor and delivery of the fetus (e.g., during the first and/or second stage of labor) because it is difficult to assess fetal health during the labor and delivery process.

We claim:

1. A method for determining an indication of a fetal venous hemoglobin oxygen saturation level comprising:
receiving, by a processor, a digital signal corresponding to an optical signal emanating from a pregnant mammal's abdomen;
determining, by the processor, whether a fetal pulsatile signal is present in the digital signal;
determining, by the processor, an indication of a fetal venous hemoglobin oxygen saturation level responsively to a determination that the fetal pulsatile signal is present in the digital signal; and
communicating, by the processor, the indication of the fetal venous hemoglobin oxygen saturation level to a user.

2. The method of claim 1, further comprising:
processing, by the processor, the digital signal to isolate a fetal venous signal prior to determining the indication of the fetal venous hemoglobin oxygen saturation level, wherein the determining the indication of the fetal venous hemoglobin oxygen saturation level is executed by analyzing an isolated fetal venous signal.

3. The method of claim 1 or 2, wherein the determining of the fetal venous oximetry level comprises performing, by the processor, a trend analysis that determines relative changes in the fetal venous oximetry level over time.

4. The method of claim 1, 2, or 3, wherein the determining the indication of the fetal venous hemoglobin oxygen saturation level comprises:
analyzing, by the processor, the digital signal; and
comparing, by the processor, a result of the analysis with a threshold value for the fetal venous hemoglobin oxygen saturation level, wherein the indication of the fetal venous hemoglobin oxygen saturation level includes a result of the comparison to the user.

5. The method of any of claims 1-4, further comprising:
receiving, by the processor, an indication of changes to a maternal venous signal included in the digital signal over a period of time;
processing, by the processor, the digital signal to remove a maternal venous component of the digital signal, thereby generating a processed digital signal that includes a fetal venous signal;
analyzing, by the processor, the processed digital signal to determine changes in the fetal venous signal of the processed digital signal over the period of time;
determining, by the processor, a trend of changes to the fetal venous signal over time; and
providing, by the processor, the trend to the user.

6. The method of any of claims 1-5, further comprising:
analyzing, by the processor, the digital signal to determine changes in the composite venous signal over a time period;
determining, by the processor, a trend of changes to the composite venous signal over time; and
providing, by the processor, the trend to the user.

7. The method of any of claims 1-6, wherein the determining of whether a fetal pulsatile signal is present in the digital signal includes determining whether an intensity of the fetal pulsatile signal is above a threshold intensity value.

8. The method of any of claims 1-7, wherein the optical signal is obtained transabdominally.

9. The method of any of claims 1-8, wherein the fetal venous hemoglobin oxygen saturation level is a fetal DC hemoglobin oxygen saturation level.

10. The method of any of claims 1-9, further comprising:
receiving, by the processor, a maternal digital signal corresponding an optical signal incident only on maternal tissue;
processing, by the processor, the digital signal to remove the maternal digital signal, thereby generating a fetal digital signal, wherein the determining of the indication of a fetal venous hemoglobin oxygen saturation level includes analyzing the fetal digital signal.

11. The method of claim 10, wherein the maternal digital signal is a short separation signal.

12. A method comprising:
receiving, by a processor, a composite DC signal from a photodetector communicatively coupled to the processor, the composite DC signal corresponding, by the processor, to an optical signal incident upon an abdomen of a pregnant mammal and a fetus contained therein that has emanated from the pregnant mammal's abdomen and been detected by the photodetector;
analyzing, by the processor, the composite DC signal;
determining, by the processor, an indication of fetal hemoglobin oxygen saturation level responsively to a result of the analysis of the composite DC signal; and
facilitating, by the processor, provision of the indication to a user.

13. The method of claim 10, wherein the analysis of the composite DC signal comprises performing, by the processor, a trend analysis that determines relative changes in the composite DC signal over time.

14. The method of claim 10 or 11, wherein the analysis of the composite DC signal comprises comparing, by the processor, a result of the analysis with a threshold value for the composite DC signal, wherein the indication includes a result of the comparison.

15. The method of any of claims 10-12, further comprising:
receiving, by the processor, an indication of changes to a maternal DC signal over a period of time;
processing, by the processor, the composite DC signal to remove changes to a maternal component of the composite DC signal, thereby generating a processed composite DC signal;
analyzing, by the processor, the processed composite DC signal to determine changes in the processed composite DC signal over the period of time;
determining, by the processor, a trend of changes to the processed composite DC signal over time; and
providing, by the processor, the trend to the user.

16. The method of any of claims 10-13, further comprising:
analyzing, by the processor, the composite DC signal to determine changes in the composite DC signal over a time period;
determining, by the processor, a trend of changes to the composite DC signal over time; and
providing, by the processor, the trend to the user.

17. The method of any of claims 10-14, further comprising:
receiving, by the processor, an indication of a fetal AC signal prior to receipt of the composite DC signal; and
determining, by the processor, whether an intensity of the fetal AC signal is above a threshold intensity value, wherein analysis of the composite DC signal is responsive to a determination that the intensity of the fetal AC signal is above the threshold intensity value.

18. The method of any of claims 10-15, wherein the composite DC signal is obtained transabdominally.

19. A method comprising:
generating, by a processor, a modeled composite DC signal, the modeled composite DC signal corresponding, by the processor, to a modeled optical signal incident upon an abdomen of a pregnant mammal and a fetus contained therein that has emanated from the pregnant mammal's abdomen and been detected;
receiving, by the processor, a measured composite DC signal from a photodetector communicatively coupled to the processor, the composite DC signal corresponding, by the processor, to an optical signal incident upon an abdomen of a pregnant mammal and a fetus contained therein that has emanated from the pregnant mammal's abdomen and been detected by the photodetector;
comparing, by the processor, the modeled composite DC signal and the measured composite DC signal;
determining, by the processor, an indication of fetal hemoglobin oxygen saturation level responsively to a result of the comparing; and
facilitating, by the processor, provision of the indication to a user.

20. The method of claim 17, further comprising:
receiving, by the processor, an indication of a fetal AC signal prior to receipt of the composite DC signal; and
determining, by the processor, whether an intensity of the fetal AC signal is above a threshold intensity value, wherein analysis of the composite DC signal is responsive to a determination that the intensity of the fetal AC signal is above the threshold intensity value.

21. A system comprising:
a processor communicatively coupled to a memory; and
the memory with a set of instructions stored therein, which when executed by the processor, cause the processor to:
receive a digital signal corresponding to an optical signal emanating from a pregnant mammal's abdomen;
determine whether a fetal pulsatile signal is present in the digital signal;
determine an indication of a fetal venous hemoglobin oxygen saturation level responsively to a determination that the fetal pulsatile signal is present in the digital signal; and
communicate the indication of the fetal venous hemoglobin oxygen saturation level to a user.

22. The system of claim 21, wherein the set of instructions, which when executed by the processor cause the processor to:
process the digital signal to isolate a fetal venous signal prior to determining the indication of the fetal venous hemoglobin oxygen saturation level, wherein the determining the indication of the fetal venous hemoglobin oxygen saturation level is executed by analyzing an isolated fetal venous signal.

23. The system of claim 21 or 22, wherein the set of instructions, which when executed by the processor cause the processor to:
analyze the digital signal; and
compare a result of the analysis with a threshold value for the fetal venous hemoglobin oxygen saturation level, wherein the indication of the fetal venous hemoglobin oxygen saturation level includes a result of the comparison to the user.

24. The system of any of claims 21-23, wherein the set of instructions, which when executed by the processor cause the processor to:
receive an indication of changes to a maternal venous signal included in the digital signal over a period of time;
process the digital signal to remove a maternal venous component of the digital signal, thereby generating a processed digital signal that includes a fetal venous signal;
analyze the processed digital signal to determine changes in the fetal venous signal of the processed digital signal over the period of time;
determine a trend of changes to the fetal venous signal over time; and
provide the trend to the user.

25. The system of any of claims 21-24, wherein the set of instructions, which when executed by the processor cause the processor to:
analyze the digital signal to determine changes in the composite venous signal over a time period;
determine a trend of changes to the composite venous signal over time; and
provide the trend to the user.

26. The system of any of claims 21-25, wherein the fetal venous hemoglobin oxygen saturation level is a fetal DC hemoglobin oxygen saturation level.

27. The system of any of claims 21-26, wherein the set of instructions, which when executed by the processor cause the processor to:
receive a maternal digital signal corresponding an optical signal incident only on maternal tissue; and
process the digital signal to remove the maternal digital signal, thereby generating a fetal digital signal, wherein the determining of the indication of a fetal venous hemoglobin oxygen saturation level includes analyzing the fetal digital signal.

28. The system of claim 27, wherein the maternal digital signal is a short separation signal.

29. A system comprising:
a processor communicatively coupled to a memory; and
the memory with a set of instructions stored therein, which when executed by the processor, cause the processor to:
receive a composite DC signal from a photodetector communicatively coupled to the processor, the composite DC signal corresponding to an optical signal incident upon an abdomen of a pregnant mammal and a fetus contained therein that has emanated from the pregnant mammal's abdomen and been detected by the photodetector;
analyze the composite DC signal;
determine an indication of fetal hemoglobin oxygen saturation level responsively to a result of the analysis of the composite DC signal; and
facilitate provision of the indication to a user.

30. The system of claim 29, wherein the analysis of the composite DC signal comprises performing a trend analysis that determines relative changes in the composite DC signal over time.

31. The system of claim 29 or 30, wherein the analysis of the composite DC signal comprises comparing a result of the analysis with a threshold value for the composite DC signal, wherein the indication includes a result of the comparison.

32. The system of any of claims 29-31, wherein the set of instructions, which when executed by the processor cause the processor to:
receive an indication of changes to a maternal DC signal over a period of time;
process the composite DC signal to remove changes to a maternal component of the composite DC signal, thereby generating a processed composite DC signal;
analyze the processed composite DC signal to determine changes in the processed composite DC signal over the period of time;
determine a trend of changes to the processed composite DC signal over time; and
provide the trend to the user.

33. The system of any of claims 29-32, wherein the set of instructions, which when executed by the processor cause the processor to:
analyze the composite DC signal to determine changes in the composite DC signal over a time period;
determine a trend of changes to the composite DC signal over time; and
provide the trend to the user.

34. The system of any of claims 29-33, wherein the set of instructions, which when executed by the processor cause the processor to:
receive an indication of a fetal AC signal prior to receipt of the composite DC signal; and
determine whether an intensity of the fetal AC signal is above a threshold intensity value, wherein analysis of the composite DC signal is responsive to a determination that the intensity of the fetal AC signal is above the threshold intensity value.

35. The system of any of claims 29-24, wherein the composite DC signal is obtained transabdominally.

36. A method comprising:
generate a modeled composite DC signal, the modeled composite DC signal corresponding to a modeled optical signal incident upon an abdomen of a pregnant mammal and a fetus contained therein that has emanated from the pregnant mammal's abdomen and been detected;
receive a measured composite DC signal from a photodetector communicatively coupled to the processor, the composite DC signal corresponds to an optical signal incident upon an abdomen of a pregnant mammal and a fetus contained therein that has emanated from the pregnant mammal's abdomen and been detected by the photodetector;
compare the modeled composite DC signal and the measured composite DC signal;
determine an indication of fetal hemoglobin oxygen saturation level responsively to a result of the comparing; and
facilitate provision of the indication to a user.

37. The system of claim 36, wherein the set of instructions, which when executed by the processor cause the processor to:
receive an indication of a fetal AC signal prior to receipt of the composite DC signal; and
determine whether an intensity of the fetal AC signal is above a threshold intensity value, wherein analysis of the composite DC signal is responsive to a determination that the intensity of the fetal AC signal is above the threshold intensity value.
